(19) United States
(12) Patent Application Publication
(10) Pub. No.: US 2024/0023969 A1
(43) Pub. Date: Jan. 25, 2024

(54) IMPLANTABLE MEDICAL DEVICE WITH PRIMARY COVERING AND SECONDARY COVERING

(71) Applicant: BOSTON SCIENTIFIC SCIMED, INC., Maple Grove, MN (US)

(72) Inventors: Brian Joseph Tischler, Shoreview, MN (US); Thyna Michael Chau, Rochester, MN (US); Joshua Mark Inouye, Brooklyn Park, MN (US); Harishankar Natesan, Minnepolis, MN (US); Jan-Hung Chen, St. Paul, MN (US)

(73) Assignee: BOSTON SCIENTIFIC SCIMED, INC., MAPLE GROVE, MN (US)

(21) Appl. No.: 18/224,343

(22) Filed: Jul. 20, 2023

Related U.S. Application Data
(60) Provisional application No. 63/391,080, filed on Jul. 21, 2022.

(51) Int. Cl. A61B 17/12

(52) U.S. Cl. CPC A61B 17/12122; A61B 17/12145; A61B 17/1215

(57) ABSTRACT

An implantable medical device such as but not limited to a left atrial appendage closure (LAAC) device includes an expandable frame that is movable between a collapsed configuration for delivery and an expanded configuration for deployment, the expandable frame including a proximal portion, a distal portion and an intervening intermediate portion. The expandable frame includes a plurality of anchors extending about a periphery of the expandable frame. A primary covering extends over the proximal portion of the expandable frame and may interact with the plurality of anchors. A secondary covering extends over part of the expandable frame.

### CROSS REFERENCE TO RELATED APPLICATIONS

This application claims the benefit of priority of U.S. Provisional Application No. 63/391,080 filed Jul. 21, 2022, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an implantable medical device. The implantable medical device includes an expandable frame defining a profile of the implantable medical device, the expandable frame movable between a collapsed configuration for delivery and an expanded configuration for deployment, the expandable frame including a proximal portion, a distal portion and an intervening intermediate portion. A primary covering extends over the proximal portion of the expandable frame. A secondary covering extends over parts of the expandable frame not covered by the primary covering.

Alternatively or additionally, the secondary covering may extend over the intermediate portion of the expandable frame.

Alternatively or additionally, the secondary covering may extend over the distal portion of the expandable frame.

Alternatively or additionally, the expandable frame may include a plurality of proximal anchors disposed about a first periphery of the expandable frame.

Alternatively or additionally, the expandable frame may include a plurality of distal anchors disposed about a second periphery of the expandable frame, the second periphery displaced axially from the first periphery.

Alternatively or additionally, the primary covering may be secured in place relative to the expandable frame via interactions between the primary covering and the plurality of proximal anchors.

Alternatively or additionally, the secondary covering may be secured in place relative to the expandable frame via interactions between the secondary covering and the plurality of distal anchors.

Alternatively or additionally, the secondary covering may be secured in place relative to the expandable frame via sutures extending between the secondary covering and the expandable frame.

Alternatively or additionally, the secondary covering may be secured in place relative to the expandable frame via sutures extending between the primary covering and the secondary covering.

Alternatively or additionally, the primary covering may have one or more first properties and the secondary covering may have one or more second properties that are different from the one or more first properties.

Alternatively or additionally, the implantable medical device may include an LAAC (left atrial appendage closure) device.

Another example may be found in a left atrial appendage closure (LAAC) device. The LAAC device includes an expandable frame defining a profile of the LAAC, the expandable frame movable between a collapsed configuration for delivery and an expanded configuration for deployment, the expandable frame including a proximal portion, a distal portion and an intervening intermediate portion. The expandable frame includes a plurality of anchors extending about a periphery of the expandable frame. A primary covering extends over the proximal portion of the expandable frame and interacting with the plurality of anchors. A secondary covering extends over part of the expandable frame.

Alternatively or additionally, the secondary covering may also interact with the plurality of anchors.

Alternatively or additionally, the secondary covering may be secured in place relative to the expandable frame via sutures extending between the secondary covering and the expandable frame.

Alternatively or additionally, the secondary covering may be secured in place relative to the expandable frame via sutures extending between the primary covering and the secondary covering.

Alternatively or additionally, the primary covering may have one or more first properties and the secondary covering may have one or more second properties that are different from the one or more first properties.

Alternatively or additionally, the secondary covering may extend over the intermediate portion and/or the distal portion of the expandable frame.

Alternatively or additionally, the primary covering may also extend over the intermediate portion of the intermediate frame, and the secondary covering may extend over the proximal portion of the intermediate frame, thereby covering part of the primary covering.

Another example may be found in a left atrial appendage closure (LAAC) device. The LAAC device includes an expandable frame defining a profile of the LAAC, the expandable frame movable between a collapsed configuration for delivery and an expanded configuration for deployment, the expandable frame including a proximal portion, a distal portion and an intervening intermediate portion. A proximal covering extends over the proximal portion of the expandable frame and the intermediate portion of the expandable frame. A distal covering may be secured relative to the expandable frame proximate a distal periphery of the proximal covering, the distal covering including segments of multiple thicknesses in order to enhance sealing between the LAAC device and tissue proximate the LAAC device post-deployment.

Alternatively or additionally, the proximal covering may further include segments of multiple thicknesses in order to enhance sealing between the LAAC device and tissue proximate the LAAC device post-deployment.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial cross-sectional view of a left atrial appendage

FIGS. 2 and 3 are side views of an illustrative system for occluding a left atrial appendage;

FIG. 4 is a perspective view of an illustrative expandable framework for a left atrial appendage closure device;

FIG. 5 is a perspective view of a portion of the illustrative expandable framework of FIG. 4;

FIG. 6 is a perspective view of an illustrative left atrial appendage closure (LAAC) device;

FIG. 7 is a perspective view of an illustrative LAAC device;

FIG. 8 is a perspective view of an illustrative LAAC device with a first covering;

FIG. 9 is a perspective view of the illustrative LAAC device of FIG. 8, with a first covering and a second covering;

FIG. 10 is a schematic view of an illustrative LAAC device disposed within an LAA; and

FIG. 11 is a schematic view of an illustrative LAAC device disposed within an LAA.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

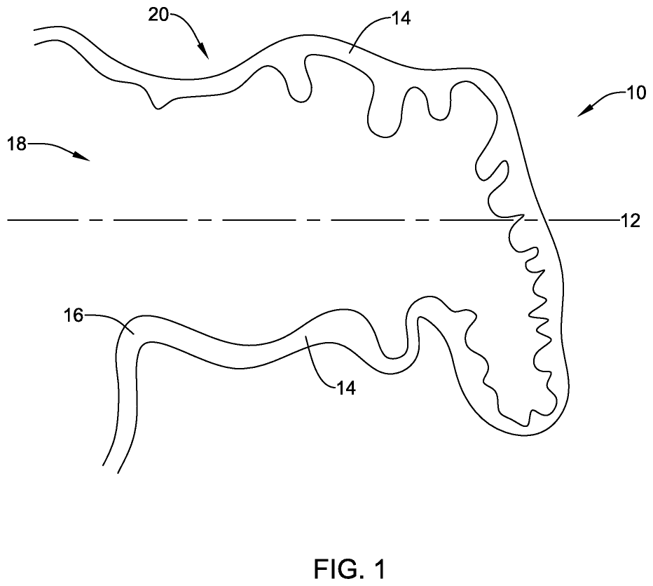

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

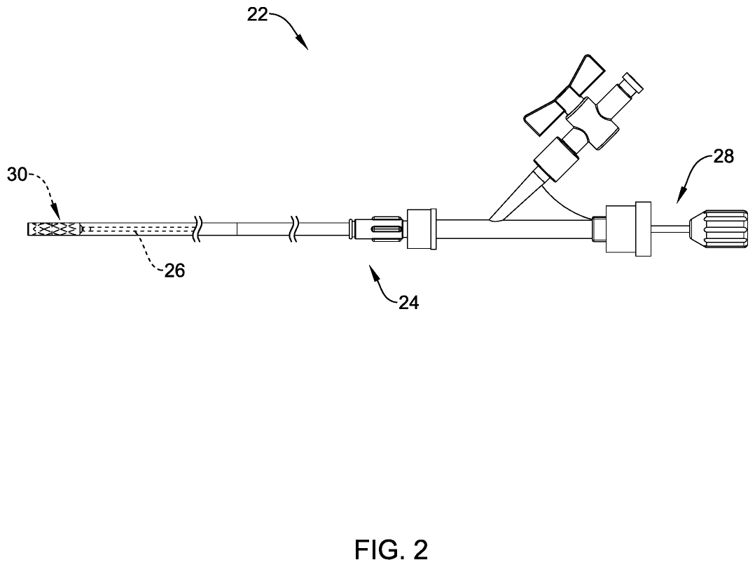

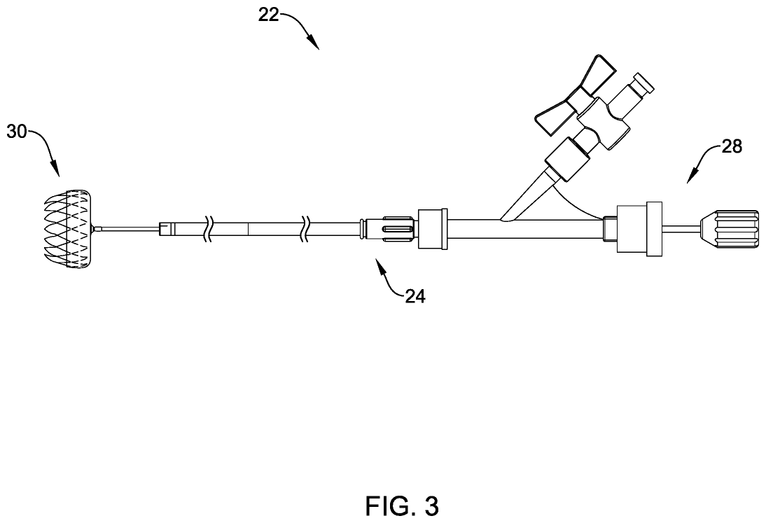

FIGS. 2 and 3 are side views of an illustrative medical device system 22. It should be noted that in any given figure, some features of the medical device system 22 may not be shown, or may be shown schematically, for simplicity. Additional details regarding some of the components of the medical device system 22 may be illustrated in other figures in greater detail. The medical device system 22 may be used to deliver and/or deploy a variety of medical implants (e.g., a cardiovascular medical implant, an occlusive medical implant, a replacement heart valve implant, etc.) to one or more locations within the anatomy, including but not limited to, in some embodiments, the heart and/or the LAA 10. In the interest of clarity, the following discussion refers to an occlusive medical implant, but other medical implants may be used and/or considered with the medical device system 22.

The medical device system 22 may include a catheter 24 having a lumen 26 extending from a proximal opening to a distal opening, a core wire 28 slidably and/or rotatably disposed within the lumen 26, and an occlusive medical implant 30 for implantation within a left atrial appendage (such as the LAA 10) releasably attached at and/or to a distal end of the core wire 28. In some embodiments, the occlusive medical implant 30 may be considered as being a left atrial appendage closure (LAAC) device.

In some embodiments, the core wire 28 may be nonrotatably secured to the occlusive medical implant 30. In some embodiments, the core wire 28 may be rotatable relative to the occlusive medical implant 30. Other configurations are also contemplated. In some embodiments, a proximal end of the core wire 28 may extend proximally of a proximal end of the catheter 24 and/or the proximal opening of the lumen 26 for manual manipulation by a clinician or practitioner. Some suitable, but non-limiting, examples of materials for the medical device system 22, the core wire 28, and/or the catheter 24, etc., including but not limited to metallic materials, polymeric materials, etc., are discussed below. It is contemplated that any and/or all example occlusive medical implants disclosed herein may be used in accordance with and/or be associated with the medical device system 22 described above.

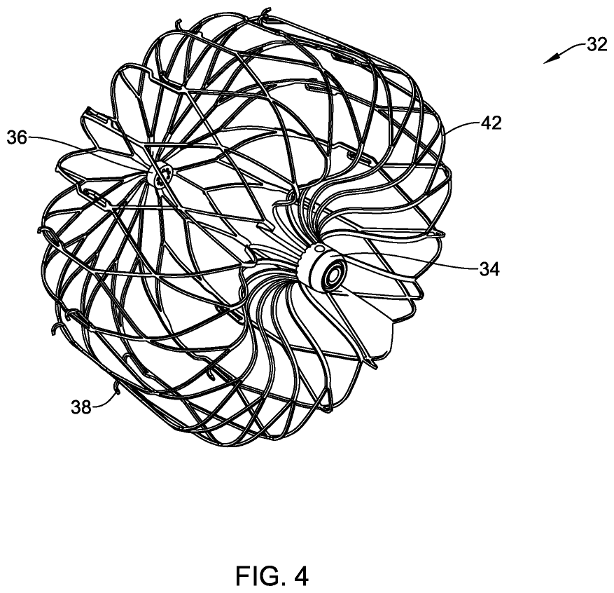

The occlusive medical implant 30 may include an expandable framework 32 (e.g., FIG. 4) configured to self-expand from a collapsed configuration (e.g., FIG. 2), such as when the occlusive medical implant 30 is disposed within the lumen 26 proximate the distal opening, to an expanded configuration (e.g., FIGS. 3 and 4), when the occlusive medical implant 30 is unconstrained by the catheter 24. In at least some embodiments, at least a portion of the expandable framework 32 may be mechanically expandable from the expanded configuration (e.g., FIGS. 3-4) to a securement configuration, as described herein.

FIG. 4 illustrates selected features of the expandable framework 32 in the fully unconstrained configuration. The expandable framework 32 may have a plurality of struts 42 disposed about a central longitudinal axis. In some embodiments, the plurality of struts 42 may define a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements.

The expandable framework 32 may include a proximal hub 34 and a distal hub 36. In some embodiments, the proximal hub 34 and/or the distal hub 36 may be centered on and/or coaxial with the longitudinal axis. The plurality of struts may be joined together at and/or fixedly attached to the proximal hub 34 and/or the distal hub 36. The proximal hub 34 may be configured to releasably connect, secure, and/or attach the occlusive medical implant 30 and/or the expandable framework 32 to the core wire 28. In some embodiments, the proximal hub 34 may include internal threads configured to rotatably and/or threadably engage an externally threaded distal end of the core wire 28. Other configurations for releasably securing the occlusive medical implant 30 to the core wire 30 are also contemplated. As noted herein, some features are not shown in every figure to improve clarity.

The expandable framework 32 and/or the plurality of struts may be formed and/or cut from a tubular member. In some embodiments, the expandable framework 32 and/or the plurality of struts may be integrally formed and/or cut from a unitary member. In some embodiments, the expandable framework 32 and/or the plurality of struts may be integrally formed and/or cut from a unitary tubular member and subsequently formed and/or heat set to a desired shape in the fully unconstrained configuration. In some embodiments, the expandable framework 32 and/or the plurality of struts may be integrally formed and/or cut from a unitary flat member or sheet, and then rolled or formed into a tubular structure and subsequently formed and/or heat set to the desired shape in the fully unconstrained configuration. Some exemplary means and/or methods of making and/or forming the expandable framework 32 and/or the plurality of struts include laser cutting, machining, punching, stamping, electro discharge machining (EDM), chemical dissolution, etc. Other means and/or methods are also contemplated.

As noted with respect to FIG. 1, anatomical features such as the LAA 10 may vary in size and/or shape. In some embodiments, the expandable framework 32 may be compliant and substantially conform to and/or be in sealing engagement with the shape and/or geometry of a lateral wall of the LAA 10 when deployed and/or expanded therein. In some embodiments, the occlusive medical device 30 may expand to a size, extent, or shape less than or different from the fully unconstrained configuration, as determined by the surrounding tissue and/or lateral wall of the LAA 10. In some embodiments, the expandable framework 32 may be configured to shape and/or stretch the tissue of the LAA 10 such that the lateral wall of the LAA 10 substantially conforms to an outer shape of the expandable framework 32. Other configurations are also contemplated.

In some embodiments, the expandable framework 32 may include at least one anchoring member 38 extending radially outward therefrom in the fully unconstrained configuration. In some embodiments, the expandable framework 32 may include at least one anchoring member 38 extending radially outward from the expandable framework 32. In some embodiments, the expandable framework 32 may include at least one anchoring member 38 extending radially outward from the expandable framework 32 proximate a proximal shoulder of the expandable framework 32. In some embodiments, the expandable framework 32 may include at least one anchoring member 38 extending radially outward from the expandable framework 32 proximate a midsection of the expandable framework 32. In some embodiments, the at least one anchoring member 38 may be configured to engage with the lateral wall 14 of the main body 20 of the LAA 10. In some embodiments, the at least one anchoring member 38 may be formed as J-shaped hooks having a free end extending in and/or directed toward a proximal direction with respect to the central longitudinal axis of the occlusive medical device 30 and/or the expandable framework 32. Other configurations are also contemplated.

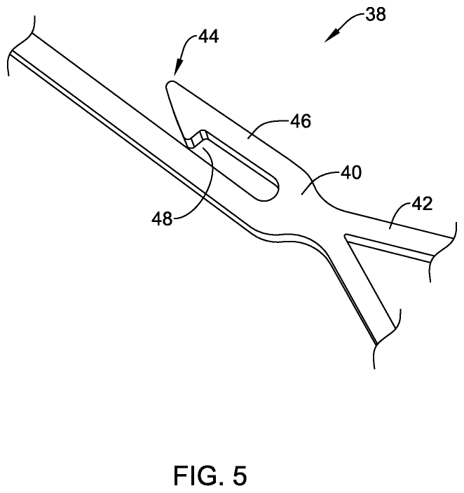

FIG. 5 provides an enlarged view of one of the anchoring members 38. As shown, the anchoring member 38 may include a base portion 40 that is attached to one of the struts 42, a tip portion 44, and a body portion 46 extending from the base portion 40 to the tip portion 44. The body portion 46 at least partially defines a void 48. In some embodiments, the tip portion 44, the body portion 46 and the void 48 may together provide an anchor for an occlusive covering that engages one or more of the anchoring members 38. In some embodiments, the tip portion 44 of each of, some of, or one of the plurality of anchoring members 38 may be radially aligned with the plurality of interconnected struts 42 (e.g., on a common circumference). In some embodiments, the body portion 46 and/or the tip portion 44 of the plurality of anchor members 38 may be oriented substantially parallel to the plurality of interconnected struts 42. In some embodiments, the tip portion 44 of each of, some of, or one of the plurality of anchor members 38 does not extend radially outward of the plurality of interconnected struts 42. As such, the plurality of anchor members 38 and/or the tip portion 44 of the plurality of anchor members 38 may be incapable of engaging with, extending into, and/or piercing native tissue(s) disposed outside of (e.g., radially outward of, etc.) the expandable framework 32 and/or the plurality of interconnected struts 42. Some suitable, but non-limiting, examples of materials for the occlusive medical implant 30, the expandable framework 32, the plurality of interconnected struts 42, etc., including but not limited to metallic materials, polymeric materials, etc., are discussed below.

In some cases, the anchoring members 38 may also be involved in helping to secure one or more occlusive coverings to the expandable framework 32 forming the occlusive medical implant 30. As shown in subsequent figures, the occlusive medical implant 30 may include one or more occlusive coverings that each cover at least a portion of the occlusive medical implant 30. In some embodiments, one or more occlusive elements may be connected to, disposed on, disposed over, disposed about, and/or disposed radially outward of at least a portion of the expandable framework 32 and/or the plurality of struts 42. In some embodiments, one or more occlusive elements may be attached to the proximal hub 34 and/or may be attached to the expandable framework 32 at the proximal hub 34. In some embodiments, one or more occlusive elements may extend radially outward from and/or may extend distally from the proximal hub 34. In some embodiments, one or more occlusive elements may be attached and/or secured to the expandable framework 32 at a plurality of discrete locations. In some embodiments, one of, some of, and/or all of the at least one anchoring members 38 may extend through an occlusive element, where present.

In some embodiments, each of the one or more occlusive elements may include a membrane, a fabric, a mesh, a tissue element, or another suitable construction. In some embodiments, each of the one or more occlusive elements may be porous. In some embodiments, the one or more occlusive elements may be non-porous. In some embodiments, the one or more occlusive elements may be permeable to selected gases and/or fluids. In some embodiments, the one or more occlusive elements may be substantially impermeable to selected gases and/or fluids, such as blood, water, etc. In some embodiments, the one or more occlusive elements may be designed, sized, and/or configured to prevent thrombus and/or embolic material from passing out of the left atrial appendage into the left atrium and/or the patient's bloodstream. In some embodiments, the one or more occlusive elements may be configured to promote endothelization after implantation, thereby effectively removing the target site (e.g., the left atrial appendage, etc.) from the patient's circulatory system. Some suitable, but non-limiting, examples of materials for the one or more occlusive elements are discussed below.

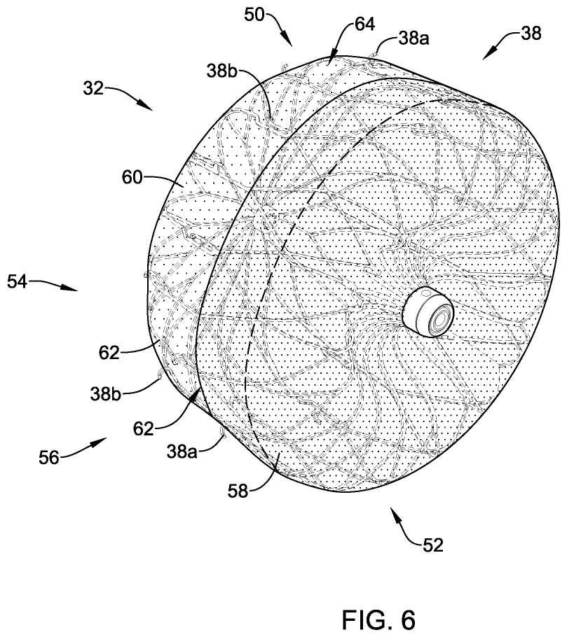

FIG. 6 is a perspective view of an illustrative LAAC device 50. The illustrative LAAC device 50 includes the expandable framework 32. The expandable framework 32 may be considered as including a proximal portion 52, a distal portion 54 and an intermediate portion 56 that is disposed between the proximal portion 52 and the distal portion 54. In some cases, as shown, the LAAC device 50 includes a primary covering 58 that extends over at least the proximal portion 52 of the expandable frame 32. The LAAC device 50 includes a secondary covering 60 that may cover parts of the expandable frame 32 that are not covered by the primary covering 58. The primary covering 58 and the secondary covering 60 are translucent, which is why the expandable frame 32 is visible underneath the primary covering 58 and the secondary covering 60. In some embodiments, as shown, the secondary covering 60 extends over the distal portion 54 of the expandable frame 32 as well as the intermediate portion 56 of the expandable frame 32. In some embodiments, the secondary covering 60 partially overlaps the primary covering 58. In some embodiments, the primary covering 58 partially overlaps the secondary covering 60, which can help enable recapture of the LAAC device 50 without impacting the edge of the secondary covering 60 as that edge is protected by the primary covering 58. The primary covering 58 and the secondary covering 60 may be considered as being examples of occlusive coverings.

The LAAC device 50 includes a number of anchoring members 38. In some embodiments, the anchoring members 38 may be considered as being divided into a plurality of proximal anchoring members 38a that are disposed about a first periphery 62 of the expandable frame 32 extending circumferentially all the way around the expandable frame 32 and a plurality of distal anchoring members 38b that are disposed about a second periphery 64 of the expandable frame 32 extending circumferentially all the way around the expandable frame 32. The second periphery 64 may be considered as being axially spaced from the first periphery 62. The relative distance between the first periphery 62 and the second periphery 64 may be varied, for example. It will be appreciated that the proximal anchoring members 38a are disposed all along the first periphery 62 and the distal anchoring members 38b are disposed all along the second periphery 64, even though only several of the proximal anchoring members 38a and several of the distal anchoring members 38b are labeled.

In some cases, the primary covering 58 may be secured in place relative to the expandable frame 32 via interactions between the primary covering 58 and at least some of the proximal anchoring members 38a. In some embodiments, interactions between the primary covering 58 and at least some of the proximal anchoring members 38a may include the proximal anchoring members 38a extending through the primary covering 58. In some embodiments, interactions between the primary covering 58 and at least some of the proximal anchoring members 38a may include the primary covering 58 being captured within the voids 48 that are at least partially defined by the body portions 46 (FIG. 5). In some cases, the primary covering 58 may also interact with at least some of the distal anchoring members 38b, depending on how far the primary covering 58 extends over the expandable framework 32. In some cases, the secondary covering 60 may be secured in place relative to the expandable frame 32 via interactions between the secondary covering 60 and at least some of the distal anchoring members 38b.

In some embodiments, interactions between the secondary covering 60 and at least some of the distal anchoring members 38b may include the distal anchoring members 38b extending through the secondary covering 60. In some embodiments, interactions between the secondary covering 60 and at least some of the distal anchoring members 38b may include the secondary covering 60 being captured within the voids 48 that are at least partially defined by the body portions 46. In some embodiments, the secondary covering 60 may be folded up along an edge thereof in order to increase its thickness, which can help with sealing against the LAA 10. In some cases, the secondary covering 60 may also interact with at least some of the proximal anchoring members 38a, depending on how far the secondary covering 60 extends over the expandable framework 32. In some cases, the primary covering 58 and the secondary covering 60 may be secured in place relative to the expandable framework 32 by virtue of interactions with the same anchoring members 38, such as either the proximal anchoring members 38a or the distal anchoring members 38b.

In some embodiments, the primary covering 58 may be secured in place relative to the expandable frame 32 via sutures (not shown) that extend between the primary covering 58 and the expandable frame 32. In some embodiments, the secondary covering 60 may be secured in place relative to the expandable frame 32 via sutures (not shown) that extend between the secondary covering 60 and the expandable frame 32. In some embodiments, the secondary covering 60 may be secured in place relative to the expandable frame 32 via sutures (not shown) that extend between the secondary covering 60 and the primary covering 58. In some embodiments, adhesives may be used instead of, or in addition to, sutures. In some embodiments, the primary covering 58 may be secured in place relative to the expandable frame 32 via interactions between the primary covering 58 and at least some of the anchoring members 38 as well as being sutured to the expandable frame 32. In some embodiments, the secondary covering 60 may be secured in place relative to the expandable frame 32 via interactions between the secondary covering 60 and at least some of the anchoring members 38 as well as being sutured to the expandable frame 32 and/or being sutured to the primary covering 58. In some embodiments, adhesives may be used instead of, or in addition to, sutures.

In some embodiments, the secondary covering 60 may be adapted to take up relatively less room when the LAAC device 50 is disposed within a delivery device such as but not limited to the medical device system 22. In some embodiments, the primary covering 58 may have one or more first properties and the secondary covering 60 may have one or more second properties that are different from the one or more first properties. As an example, the primary covering 58 may have a first thickness and the secondary covering 60 may have a second thickness that is less than the first thickness. As another example, the primary covering 58 may have a first density and the second covering 60 may have a second density that is less than the first density. As another example, the primary covering 58 may be a knitted, woven or braided fabric formed from a thread having a first diameter and the secondary covering 60 may be a woven or braided fabric formed from a thread having a second diameter that is less than the first diameter.

In some embodiments, the primary covering 58 and the secondary covering 60 may each be formed of a knitted, woven or braided fabric available commercially from Aran Biomedical. In some embodiments, the primary covering 58 may be a fabric having a Denier rating of 15 to 20, or perhaps 20 to 25, while the secondary covering 60 may be a fabric having a Denier rating of 10 to 15. In some embodiments, the secondary covering 60 may be about twenty five percent lighter than the primary covering 58. It will be appreciated that the Denier rating of a fabric is a unit that pertains to the weight and thickness of the individual threads used in the fabric. Fabric with a higher Denier rating will be thicker and heavier than the same fabric with a lower Denier rating.

In some embodiments, the primary covering 58 may have a first knit pattern and the second covering 60 may have a second knit pattern that is different from the first knit pattern. The primary covering 58 may have a first pore size while the secondary covering 60 may have a different pore size. The secondary member 60 may be knitted in a honeycomb pattern, for example. In some embodiments, the primary covering 58 and the secondary covering 60 may vary in one or more of knit pattern, material and post-knit processing in order to achieve desired pore size and mechanical properties of the primary covering 58 and the secondary covering 60. In some embodiments, the primary covering 58 may have a first elasticity while the secondary covering 60 may have a different elasticity. The primary covering 58 may have a greater elasticity than the secondary covering 60. In some embodiments, the primary covering 58 may have a reduced elasticity relative to the secondary covering 60.

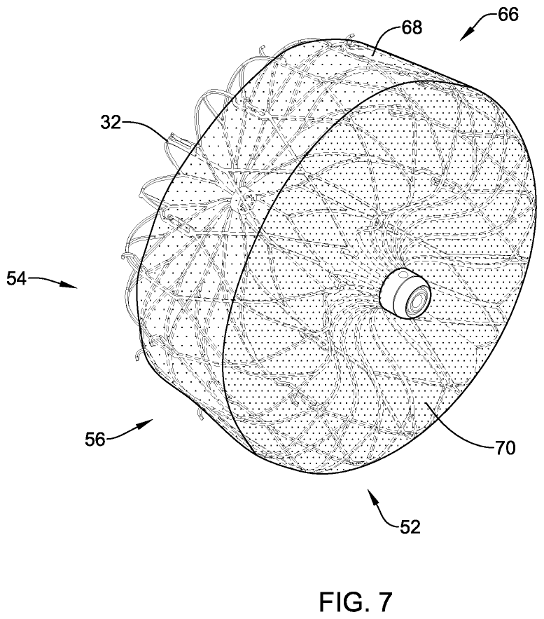

FIG. 7 is a perspective view of an illustrative LAAC device 66. The illustrative LAAC device 66 includes the expandable framework 32. As before, the expandable framework 32 includes the proximal portion 52, the distal portion 54 and the intermediate portion 56. The LAAC device 66 includes a primary covering 68 that covers the proximal portion 52 and the intermediate portion 56. The primary covering 68 may be considered as covering at least part of the distal portion 54 as well. The LAAC device 66 also includes a secondary covering 70 that overlays part of the primary covering 68 and is disposed over the proximal portion 52 of the expandable framework 32. In some embodiments, the secondary covering 70 may be coated differently, or may have a different porosity relative to the primary covering 68. In some embodiments, the secondary covering 70 may be formed of a different material type relative to the primary covering 68. In some embodiments, the primary covering 68 is on the outside and the secondary covering 70 is on the inside, such that only an edge of the secondary covering 70 is only accessible from an interior of the expandable frame 32.

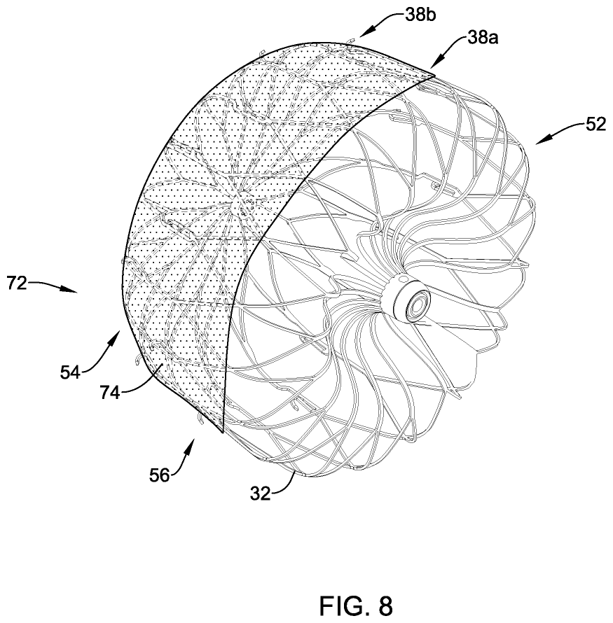

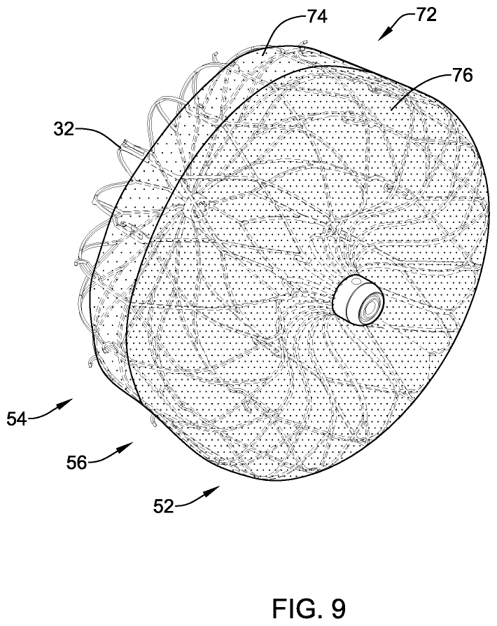

FIGS. 8 and 9 are views showing assembly of an illustrative LAAC device 72. The illustrative LAAC device 72 includes the expandable framework 32. As before, the expandable framework 32 includes the proximal portion 52, the distal portion 54 and the intermediate portion 56. As shown in FIG. 8, the LAAC device 72 includes a secondary covering 74 that is disposed over part of the distal portion 54 of the expandable frame 32, but does not cover all of the distal portion 54 of the expandable frame 54.

In FIG. 9, a primary covering 76 has been added. The secondary covering 74 may be considered as being adapted to effectively extend the sealing abilities of the LAAC 72, particularly if the LAAC 72 is deployed in a position that is too far proximal relative to the mouth 18 of the LAA 10. In some embodiments, the secondary covering 74 is secured in position relative to the expandable framework 32 via interactions with at least some of the distal anchoring members 38b and the primary covering 76 is secured in position relative to the expandable framework 32 via interactions with at least some of the proximal anchoring members 38a. In some embodiments, the secondary covering 74 may be secured relative to the expandable framework 32 via interactions with at least some of the distal anchoring members 38b and at least some of the proximal anchoring members 38a. In some embodiments, the primary covering 76 may be anchored over at least part of the secondary covering 74, thereby helping to anchor the secondary cover 74 in position.

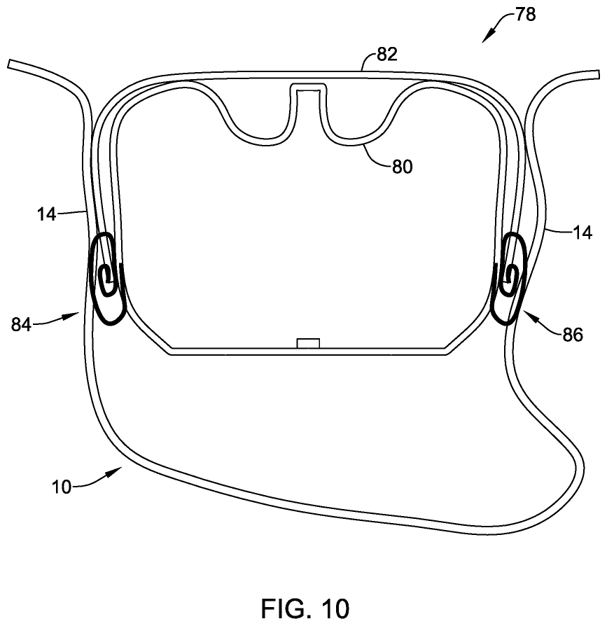

In some embodiments, the secondary covering may help with sealing by providing a bunched up, rolled up or folded up section of fabric that helps to fill any gaps between the LAAC and the side walls 14 of the LAA 10. FIG. 10 is a schematic view of an illustrative LAAC device 78 shown disposed within a representation of the LAA 10. The illustrative LAAC device 78 includes an expandable framework 80 that may be considered as being an example of the expandable framework 32. The LAAC device 78 includes a primary covering 82 that covers at least a proximal part of the expandable framework 80. The LAAC device 78 also includes a secondary covering 84 that, by virtue of a roll-up process, is coiled up into a coil configuration 86, thereby providing multiple thicknesses of material. The roll-up process is accomplished during the deployment phase. Once the secondary covering 84 is exposed from the medical device system 22, the fabric coils up as a result of coiled metallic strands that are embedded into the fabric. Alternatively, the fabric has a coiled shape by nature but is flattened out when loaded in the medical device system 22. As shown, the coil configuration 86 fills up space between the LAAC 78 and the side walls 14 of the LAA 10, thereby helping to seal against the LAA 10.

In some embodiments, the secondary covering 84 may be made from a shape-memory polymer. In some embodiments, the secondary covering 84 may include thin strips of a shape-memory material that are woven into the fabric of the secondary covering 84. The thin strips of shape-memory material may be Nitinol, although other shape-memory materials are contemplated. In some embodiments, recapture helps to straighten out the secondary covering 84 to prevent bunch-up when the LAAC 78 is packed into the medical device system 22 for subsequent delivery. In some embodiments, fabric “rollback” phenomenon that occurs when nominal fabric is processed with certain heat set times and is handled to stretch the outer lip during assembly, for example.

In some embodiments, instead of just causing the secondary covering 84 to roll up, the primary covering 82 may also be adapted to roll up. A distal edge of both the primary covering 82 and the secondary covering 84 may include thin strips of a shape memory material such as Nitinol woven into the primary covering 82 and the secondary covering 84. In some embodiments, both the primary covering 82 and the secondary covering 84 may include a shape memory polymer, or may be processed to take advantage of natural rollback phenomenon. As each fabric portion has an alternating spot to roll up between each row of anchors 38, the roll-up continues all the way around the LAAC 78, providing additional sealing all the way around the LAAC 78. Again, recapture may straighten out the distal fabric to avoid bunch-up in the medical device system 22.

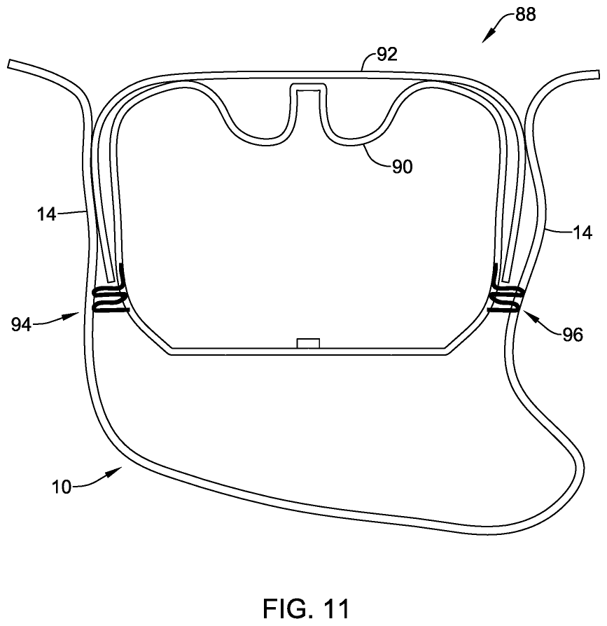

FIG. 11 is a schematic view of an illustrative LAAC device 88 shown disposed within a representation of the LAA 10. The illustrative LAAC device 88 includes an expandable framework 90 that may be considered as being an example of the expandable framework 32. The LAAC device 88 includes a primary covering 92 that covers at least a proximal part of the expandable framework 90. The LAAC device 88 also includes a secondary covering 94 that is adapted to revert to a bunched up configuration 96, thereby providing multiple thicknesses of material. As shown, the bunched up configuration 96 fills up space between the LAAC 88 and the side walls 14 of the LAA 10, thereby helping to seal against the LAA 10. The secondary covering 94 may be formed of a shape memory polymer, for example, or may include thin Nitinol strands that are shape set to the bunched up configuration 96. The secondary covering 94 may be straightened out when recaptured, to avoid bunching up in the medical device system 22. In some embodiments, radiopaque materials may be sewn or folded into the fabric to help identify key components of the LAAC 88 by X-ray during the implantation process.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device, comprising:
an expandable frame defining a profile of the implantable medical device, the expandable frame movable between a collapsed configuration for delivery and an expanded configuration for deployment, the expandable frame including a proximal portion, a distal portion and an intervening intermediate portion;
a primary covering extending over the proximal portion of the expandable frame; and
a secondary covering extending over parts of the expandable frame not covered by the primary covering.

2. The implantable medical device of claim 1, wherein the secondary covering extends over the intermediate portion of the expandable frame.

3. The implantable medical device of claim 1, wherein the secondary covering extends over the distal portion of the expandable frame.

4. The implantable medical device of claim 1, wherein the expandable frame comprises a plurality of proximal anchors disposed about a first periphery of the expandable frame.

5. The implantable medical device of claim 4, wherein the expandable frame comprises a plurality of distal anchors disposed about a second periphery of the expandable frame, the second periphery displaced axially from the first periphery.

6. The implantable medical device of claim 5, wherein the primary covering is secured in place relative to the expandable frame via interactions between the primary covering and the plurality of proximal anchors.

7. The implantable medical device of claim 5, wherein the secondary covering is secured in place relative to the expandable frame via interactions between the secondary covering and the plurality of distal anchors.

8. The implantable medical device of claim 1, wherein the secondary covering is secured in place relative to the expandable frame via sutures extending between the secondary covering and the expandable frame.

9. The implantable medical device of claim 1, wherein the secondary covering is secured in place relative to the expandable frame via sutures extending between the primary covering and the secondary covering.

10. The implantable medical device of claim 1, wherein the primary covering has one or more first properties and the secondary covering has one or more second properties that are different from the one or more first properties.

11. The implantable medical device of claim 1, comprising an LAAC (left atrial appendage closure) device.

12. A left atrial appendage closure (LAAC) device, comprising:
an expandable frame defining a profile of the LAAC, the expandable frame movable between a collapsed configuration for delivery and an expanded configuration for deployment, the expandable frame including a proximal portion, a distal portion and an intervening intermediate portion;
the expandable frame including a plurality of anchors extending about a periphery of the expandable frame;
a primary covering extending over the proximal portion of the expandable frame and interacting with the plurality of anchors; and
a secondary covering extending over part of the expandable frame.

13. The LAAC device of claim 12, wherein the secondary covering also interacts with the plurality of anchors.

14. The LAAC device of claim 12, wherein the secondary covering is secured in place relative to the expandable frame via sutures extending between the secondary covering and the expandable frame.

15. The LAAC device of claim 12, wherein the secondary covering is secured in place relative to the expandable frame via sutures extending between the primary covering and the secondary covering.

16. The LAAC device of claim 12, wherein the primary covering has one or more first properties and the secondary covering has a one or more second properties that are different from the one or more first properties.

17. The LAAC device of claim 12, wherein the secondary covering extends over the intermediate portion and/or the distal portion of the expandable frame.

18. The LAAC device of claim 12, wherein:
the primary covering also extends over the intermediate portion of the intermediate frame; and
the secondary covering extends over the proximal portion of the intermediate frame, thereby covering part of the primary covering.

19. A left atrial appendage closure (LAAC) device, comprising:
an expandable frame defining a profile of the LAAC, the expandable frame movable between a collapsed configuration for delivery and an expanded configuration for deployment, the expandable frame including a proximal portion, a distal portion and an intervening intermediate portion;
a proximal covering extending over the proximal portion of the expandable frame and the intermediate portion of the expandable frame; and
a distal covering secured relative to the expandable frame proximate a distal periphery of the proximal covering, the distal covering including segments of multiple thicknesses in order to enhance sealing between the LAAC device and tissue proximate the LAAC device post-deployment.

20. The LAAC device of claim 19, wherein the proximal covering further comprises segments of multiple thicknesses in order to enhance sealing between the LAAC device and tissue proximate the LAAC device post-deployment.
